(19) United States
(12) Patent Application Publication
(10) Pub. No.: US 2004/0234460 A1
(43) Pub. Date: Nov. 25, 2004

(54) Tooth whitening compositions and methods for using the same

(76) Inventors: Jeanna Gail Tarver, Bountiful, UT (US); Benjamin Dee Card, Mapleton, UT (US); Michael Robert LaDow, Provo, UT (US); Donald Hugh Livingstone, Provo, UT (US)

Correspondence Address: Berne S. Broadbent, KIRTON & McCONKIE, Suite 1800, 60 East South Temple, Salt Lake City, UT, 84111, US

(21) Appl. No.: 10/442,559

(22) Filed: May 21, 2003

(51) Int. Cl. A61K 7/16

(52) U.S. Cl. 424/49

(57) ABSTRACT

A tooth whitening system that effectively conceals tooth discoloration within a short period of time. The system includes a single, spectrally pure dye and a carrier for applying the dye to the tooth.

### BACKGROUND OF THE INVENTION

1. Field of the Invention

The present invention relates to compositions for whitening teeth. More particularly, the present invention relates to a dye suitable for application to teeth that causes the teeth to appear whiter.

2. Background and Related Art

Tooth whitening is increasingly recognized as a relatively quick and inexpensive way to improve one's appearance. Accordingly, many compositions and methods for obtaining whiter teeth are presently known.

Many such compositions and methods, however, require professional application and are thus both costly and time consuming. Indeed, all laser and some bleaching procedures must be performed by dentists in a dental office, and can cost thousands of dollars depending on the number of teeth to be treated and the severity of their discoloration. Even some compositions capable of superficial application require professional supervision due to their extreme potency and inherent risks upon application.

Indeed, most presently known topically applied compositions for whitening teeth incorporate invasive chemical components to break down pigments internally disposed in teeth. Even when properly applied, these chemical components have been known to cause increased tooth sensitivity in some people, and moderate to severe pain in others. One theory is that increased sensitivity and pain result from the bleaching agent working its way through pores which naturally occur in teeth and into the pulp chamber. Others have posited that certain carriers can cause teeth to become somewhat desiccated or dehydrated, which can cause increased internal fluid pressure and pain within the pulp chamber.

Because of the extreme costs and risks associated with traditional bleaching compositions and techniques, less potent compositions available for home use have become increasingly popular and are widely available in over-the-counter forms. Although such compositions avoid the disadvantages of more potent bleaching systems, such home use compositions are typically less effective in removing and/or concealing tooth discoloration, and require increased frequency and duration of use.

Accordingly, what is needed is an effective, non-invasive, non-destructive tooth whitening system. Such compositions and methods for whitening teeth are disclosed and claimed herein.

### SUMMARY OF THE INVENTION

The present invention is a tooth whitening system that effectively conceals tooth discoloration within a short period of time.

An object of the present invention is to provide a tooth whitening system capable of topical application that incorporates a spectrally pure dye to effectively mask tooth discoloration.

Another object of the invention is to provide a topically applied tooth whitening system that provides maximum whitening effect without causing tooth sensitivity.

Another object of the present invention is to provide an inexpensive tooth whitening system safe for home use.

It is a further object of the present invention to provide a tooth whitening system capable of producing effective results within a short period of time from initial application.

These and other features and advantages of the present invention will be set forth or will become more fully apparent in the description that follows. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

The foregoing and other objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 is a comparative chart of ranges of wavelengths absorbed and/or reflected by dyes of particular colors;

FIG. 2 is a comparative analysis showing an absorption spectrum of a subtractive dye mixture containing red and blue pigments compared to that of a spectrally pure violet dye;

FIG. 3 is a spectral analysis of one embodiment of a dye in accordance with the present invention;

FIG. 4 is a spectral analysis of a second embodiment of a dye in accordance with the present invention;

FIG. 5 is a spectral analysis of a third embodiment of a dye in accordance with the present invention; and

FIG. 6 is a spectral analysis of a fourth embodiment of a dye in accordance with the present invention.

### DETAILED DESCRIPTION OF THE INVENTION

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

As used in this specification, the term “visible spectrum” refers to the band of electromagnetic radiation ranging from wavelengths of approximately 400 to 700 nm, corresponding to the sensitivity of the human eye. The term “white light” refers to the mixture of all wavelengths in the visible spectrum having the relative intensities produced by a body at a white heat. The term “color” indicates a wavelength of light perceived upon absorption of a portion of the visible spectrum. The term “complementary color” means a color capable of reflecting a specific wavelength of light, which wavelength results from subtracting another particular color wavelength from white light, i.e., the wavelength remaining after certain wavelengths are absorbed. A color perceived is complementary to the color absorbed. The term “peak wavelength” refers to that portion of a wavelength having significantly higher intensity relative to its immediately adjacent portions. The term “dye” refers to any FD&C dye, FD&C lake, or natural food color such as carmine, carotene, annatto, turmeric, curcumin, blueberry and other natural food colors known in the art as adapted for use in the tooth whitening system of the present invention. The term “spectrally pure dye” refers to any dye that absorbs and/or reflects a wavelength of light having a single peak wavelength within the visible spectrum.

Where all wavelengths of light corresponding to the visible spectrum are reflected, the color perceived is white. Conversely, where all wavelengths of light corresponding to the visible spectrum are absorbed, the color perceived is muddy brown to black.

A pigment or dye modifies light by absorbing a portion of the wavelengths in the visible spectrum. The light that is not absorbed by the pigment or dye is reflected. The definition of a color thus perceived may be termed either according to wavelength absorbed or wavelength reflected. For the purposes of the present invention, all wavelengths used to define the perception of a certain color will be termed according to wavelength absorbed rather than wavelength reflected.

An exemplary non-invasive tooth whitening composition capable of home use is disclosed in U.S. Pat. No. 6,030,22 to Tarver (“Tarver”). Tarver teaches a whitening composition that includes a complementary dye that, when absorbed by a tooth, causes the tooth to reflect a color of light that is whiter than the natural or initial color of light reflected by the tooth. According, to Tarver, the dye color that can blend with most off-white and yellowish-tinted teeth to yield whiter looking teeth is in a range from violet to blue-violet.

The effectiveness of the compositions disclosed in Tarver, however, is limited by the fact that the disclosed violet or blue-violet dye comprises unspecified quantities of red and blue dyes combined to create the violet to blue-violet color. In fact, the process of combining red and blue dyes only gives the appearance of a dye in a range from violet to blue-violet. Indeed, combining dyes capable of absorbing and reflecting discrete wavelengths of light results in a dye composition that, though appearing to reflect a wavelength of light intermediate the wavelengths of light initially reflected by the individual dye components, actually only continues to reflect the discrete, but now closely integrated, initially reflected wavelengths of light. The resulting color, in this case violet or blue-violet, is thus nothing more than an optical illusion.

Indeed, this phenomenon may be compared to the kind of optical illusion created by an impressionistic work of art. Many pieces of art created in the impressionistic style utilize optical blending to impart a sense of warmth to an otherwise realistic representation. Georges Seurat exemplified this art style in “A Sunday on La Grande Jatte.” With a kind of scientific precision, Seurat juxtaposed tiny dots of various colors to create, through optical blending, what Seurat considered a more luminous single-tone hue. This same method of juxtaposing colors to create color synergy is disclosed in Tarver on a molecular level, where discrete red and blue dyes are combined to appear violet.

The heterogeneous dye combination taught by Tarver may cause discoloration of surrounding oral tissues. Upon application of the dye to teeth, the gums, tongue and surrounding oral tissues may evidence a red tint due to the continued presence of iodine-containing red dye.

It has recently been discovered that the whitening effect of a single, spectrally pure violet dye is significantly superior to a combination dye as disclosed by Tarver. Further, a spectrally pure dye applied to a tooth prevents oral tissue discoloration resulting from application of a heterogeneous mixture containing red dye.

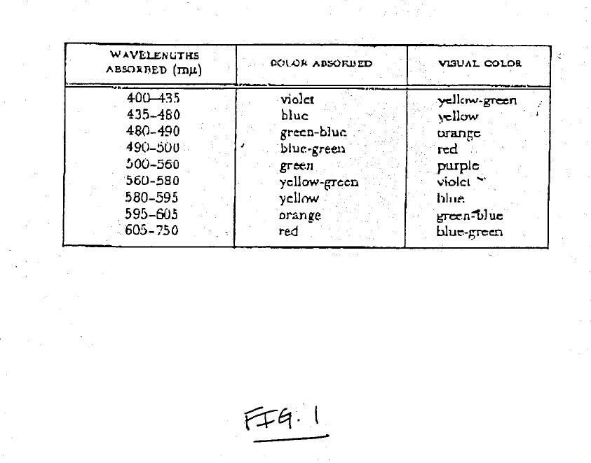

Referring to FIG. 1, the color corresponding to a wavelength of light reflected has an inverse relationship to the color corresponding to a wavelength of light absorbed. For example, a color perceived as violet is caused by absorption-of light having a wavelength in a range between 560-580 nm, corresponding to the color yellow-green. Likewise, a color perceived as yellow-green is caused by absorption of light having a wavelength in a range between 400-435 nm, corresponding to the color violet. Thus, yellow-green and violet are complementary colors.

The present invention seeks to exploit this relationship between colors by providing a dye for a tooth that absorbs a wavelength that is complementary to the wavelength initially absorbed by the tooth. In this manner, the tooth may acquire a whiter appearance.

A typical healthy tooth consists of an enamel surface and a dentin sub-layer beneath the enamel surface. The enamel surface comprises a highly calcified material that is preferably very hard and durable to allow chewing a wide variety of foods. The dentin sub-layer is also durable but considerably softer than the enamel. In addition, dentin tends to have more of a yellowish hue compared to enamel, which tends to be white. Enamel may also be somewhat clear or translucent, which may enable yellowish tints of the dentin to show through the enamel.

Depending on the thickness, opacity and whiteness of the enamel, the color reflected by a person's teeth can vary widely between various shades of white, off-white and yellow. Teeth can also accumulate plaque and tartar, which may exacerbate yellowish discoloration.

It has been found that application of a small amount of dye reflecting a wavelength corresponding to violet or bluish-violet tends to offset any yellowish tints inherently reflected by a tooth, thus producing a whiter appearance. More importantly, it has recently been discovered that use of a small amount of a spectrally pure dye having a single peak wavelength within an absorption range of 480 to 660 nm may cause an otherwise yellowish tooth to appear substantially whiter than use of a combination dye produced to appear violet or bluish-violet.

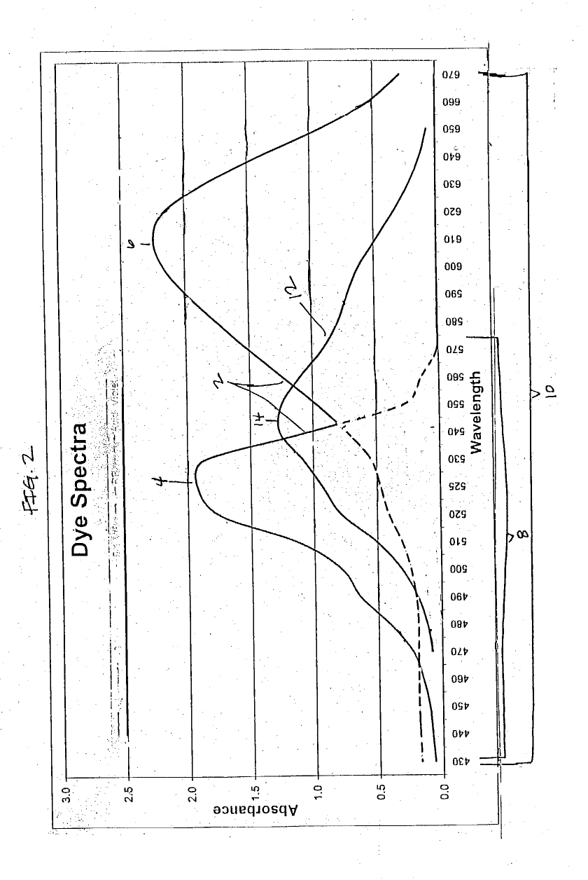

Referring now to FIG. 2, a combination of red and blue dyes combined to appear violet exhibits a spectral identity 2 having two peak wavelength absorbencies, wherein a first peak 4 corresponds to a peak wavelength absorbency unique to the red dye and a second peak 6 corresponds to a peak wavelength absorbency unique to the blue dye, respectively. The resulting wavelength 2 is thus not violet at all, but simply the sum of the red wavelength 8 and the blue wavelength 10. True violet, on the other hand, exhibits a spectrally pure identity 12 having a single peak wavelength absorbency 14 corresponding to a point intermediate the peak wavelength absorbencies 4 and 6 for red and blue.

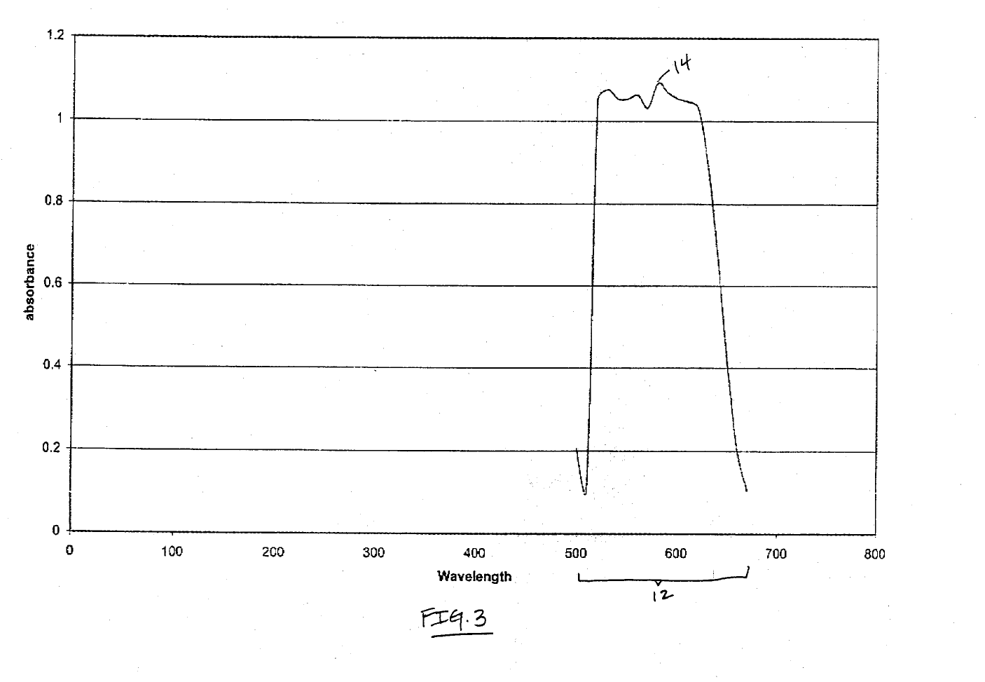

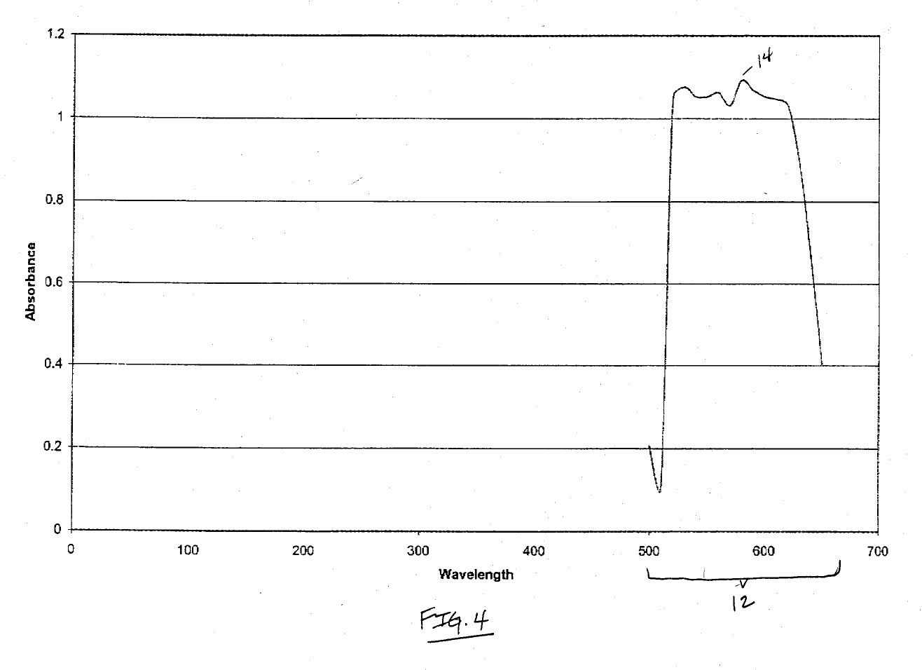

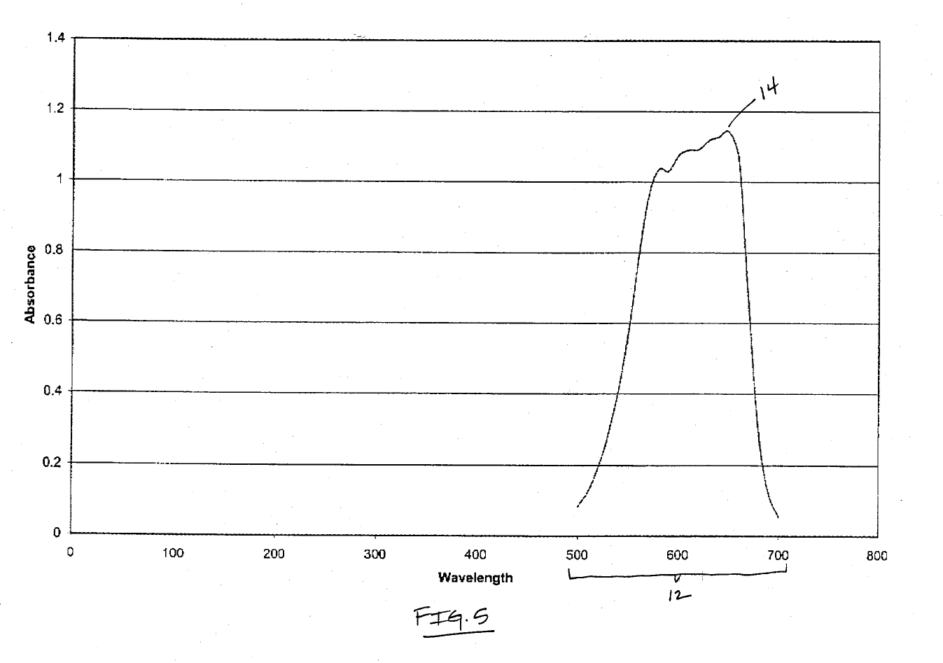

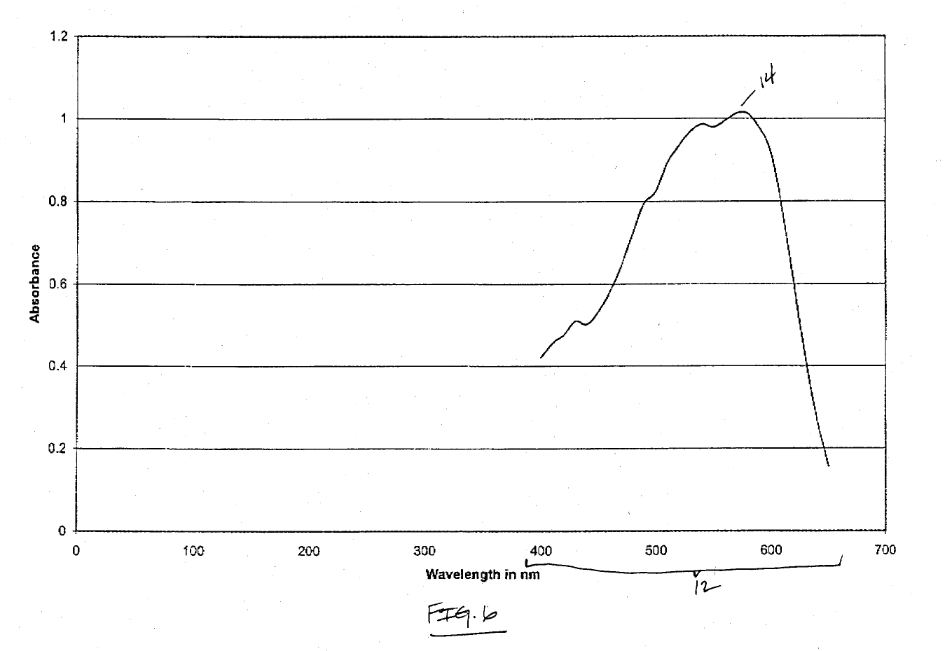

Referring now to FIGS. 3 to 6, a dye in accordance with the present invention may comprise any dye that is spectrally pure within the visible spectrum, and that has a wavelength absorbancy range from 480 to 660 nm, more preferably in a range from 500 to 620 nm, and most preferably in a range from 540 to 595 nm. A dye may comprise, for example, FD&C Red No. 3, FD&C Red No. 33, FD&C Red No. 40, FD&C Blue No. 1, FD&C Blue No. 2, FD&C Violet No. 2, FD&C Acid Violet No. 17, Carmine Red, Carmine Violet, Blueberry Additive, or any other spectrally pure artificial or natural colorant known to those in the art that exhibits a color or effect of a color having an absorbency wavelength in a range from 480 to 660 nm. The specific wavelength attributes of various dyes in accordance with the present invention are set forth in the Figures as follows: FIG. 3 corresponds to Acid Violet No. 17; FIG. 4 corresponds to FD&C Red No. 33; FIG. 5 corresponds to FD&C Blue No. 1; and FIG. 6 corresponds to Carmine 12011.

The distinction between a single, spectrally pure dye and a combination dye is of paramount importance to the effectiveness of a tooth dye as taught by the present invention. Indeed, remarkable results far superior to those formerly achieved by a combination dye such as that disclosed by Tarver have been achieved as a result of application of a single, spectrally pure dye as disclosed herein.

Moreover, because the present dye is spectrally pure, it avoids the possible negative effect of red staining typical of combination dyes. Indeed, red staining may occur where a combination dye retains a discrete chemical structure corresponding to red dye. FD&C Red No. 3 is particularly prone to exude a red staining effect due to exposed iodine molecules bordering the chemical structure.

According to one aspect of the present invention, the tooth whitening composition comprises a carrier in addition to a dye. One preferred class of carriers within the scope of the present invention comprises mouthwash and/or any hydrophilic carrier designed to wash or be sprayed onto a person's teeth. Such carriers enable the dye to be evenly and uniformly dispersed over and absorbed into a person's teeth. Moreover, hydrophilic carriers enable faster absorption of a dye by a person's teeth since such carriers are attracted by the aqueous environment surrounding teeth. By way of example and not limitation, a mouthwash or other hydrophilic carrier may comprise water, alcohols, glycerin and other polyhydric alcohols, polyols, ketones, aldehydes, carboxylic acids, carboxylic acid salts and amines.

According to another embodiment of the present invention, a gelling agent may be used as a carrier to yield a gel or more viscous liquid for application to a person's teeth. Such gelling agents may include, but are not limited to, polycarboxylic acids, polycarboxylic acid salts, polysaccharides, polysaccharide derivatives, proteins, protein derivatives, polyalkylene oxides, fumed silica and the like.

According to another embodiment of the present invention, a dye may be used in combination with a toothpaste or other dentifrice used to clean, protect and/or whiten a person's teeth. For example, the present invention may be used in combination with brush-on applications, prophylaxis pastes for professional cleaning applications, temporary plastic strips that are topically applied to teeth, sealants, applications involving curing lights, applications involving laser whitening treatments, carbomide peroxide gel whitening treatments, peroxide gel whitening treatments, or any other tooth whitening, protecting or cleaning treatment known to those in the art.

According to yet another embodiment of the present invention, a dye may be incorporated into a chewing gum such that the dye is released gradually as the gum is chewed. A gum may comprise, for example, sorbitol, gum base, mannitol, glycerin, acesulfame potassium, asparatame and flavoring.

What is claimed is:

1. A tooth whitening composition for topical application to at least one tooth, wherein, said tooth absorbs an initial wavelength of light, said composition comprising:
a single, spectrally pure dye capable of absorbing light having a wavelength that is complementary to said initial wavelength of light; and
a carrier.

2. The tooth whitening composition of claim 1, wherein said light comprises a peak wavelength in a range from 480 to 660 nm.

3. The tooth whitening composition of claim 1, wherein said light comprises a peak wavelength in a range from 480 to 500 nm.

4. The tooth whitening composition of claim 1, wherein said light comprises a peak wavelength in a range from 500 to 520 nm.

5. The tooth whitening composition of claim 1, wherein said light comprises a peak wavelength in a range from 520 to 540 nm.

6. The tooth whitening composition of claim 1, wherein said light comprises a peak wavelength in a range from 540 to 560 nm.

7. The tooth whitening composition of claim 1, wherein said light comprises a peak wavelength in a range from 560 to 580 nm.

8. The tooth whitening composition of claim 1, wherein said light comprises a peak wavelength in a range from 580 to 600 nm.

9. The tooth whitening composition of claim 1, wherein said light comprises a peak wavelength in a range from 600 to 620 nm.

10. The tooth whitening composition of claim 1, wherein said light comprises a peak wavelength in a range from 620 to 640 nm.

11. The tooth whitening composition of claim 1, wherein said light comprises a peak wavelength in a range from 640 to 660 nm.

12. The tooth whitening composition of claim 1, wherein said dye is selected from the group consisting of FD&C Red No. 3, FD&C Red No. 33, FD&C Red No. 40, FD&C Blue No. 1, FD&C Blue No. 2, FD&C Violet #2, FD&C Acid Violet No. 17, Carmine red, Carmine violet and Blueberry Additive.

13. The tooth whitening composition of claim 1, wherein said carrier is selected from the group consisting of a mouthwash, a hydrophilic solvent, a gelling agent, and a gum.

14. The tooth whitening composition of claim 13, wherein said hydrophilic solvent is selected from the group consisting of water, glycerin, alcohols, polyhydric alcohols, polyols, ketones, aldehydes, carboxylic acids, carboxylic acid salts, amines and mixtures thereof.

15. The tooth whitening composition of claim 13, wherein said gelling agent is selected from the group consisting of polycarboxylic acids, polycarboxylic acid salts, plysaccharides, polysaccharide derivatives, proteins, protein derivatives, polyalkylene oxides, fumed silica, and mixtures thereof.

16. The tooth whitening composition of claim 13, wherein said gum comprises sorbitol, gum base, mannitol, glycerin, acesulfame potassium, aspartame and flavoring.

17. A tooth whitening composition having a carrier and a dye capable of absorbing light which is complementary to an initial light absorbed by a person's tooth such that application of the dye to the person's tooth results in the tooth reflecting light that is perceived to be whiter than the light initially reflected thereby, wherein the improvement comprises:
a single, spectrally pure dye capable of absorbing light having a single peak wavelength in a range from 480 to 660 nm.

18. The tooth whitening composition of claim 17, wherein said single peak wavelength is in a range from 480 to 500 nm.

19. The tooth whitening composition of claim 17, wherein said single peak wavelength is in a range from 500 to 520 nm.

20. The tooth whitening composition of claim 17, wherein said single peak wavelength is in a range from 520 to 540 nm.

21. The tooth whitening composition of claim 17, wherein said single peak wavelength is in a range from 540 to 560 nm.

22. The tooth whitening composition of claim 17, wherein said single peak wavelength is in a range from 560 to 580 nm.

23. The tooth whitening composition of claim 17, wherein said single peak wavelength is in a range from 580 to 600 nm.

24. The tooth whitening composition of claim 17, wherein said single peak wavelength is in a range from 600 to 620 nm.

25. The tooth whitening composition of claim 17, wherein said single peak wavelength is in a range from 620 to 640 nm.

26. The tooth whitening composition of claim 17, wherein said single peak wavelength is in a range from 640 to 660 nm.

27. A method for whitening teeth including (i) contacting at least one tooth with a tooth whitening composition having a carrier and a dye capable of absorbing light which is complementary to an initial light absorbed by a person's tooth such that absorption of the dye by the person's tooth results in the tooth reflecting light that is perceived to be whiter than the light initially reflected thereby; (ii) allowing the tooth whitening composition to remain in contact with the person's tooth for a desired time period; and (iii) removing excess dye from the person's tooth, wherein the improvement comprises:
providing a dye capable of absorbing light, having a single peak wavelength in a range from 480 to 660 nm.

28. The method of claim 27, wherein said single peak wavelength is in a range from 480 to 500 nm.

29. The method of claim 27, wherein said single peak wavelength is in a range from 500 to 520 nm.

30. The method of claim 27, wherein said single peak wavelength is in a range from 520 to 540 nm.

31. The method of claim 27, wherein said single peak wavelength is in a range from 540 to 560 nm.

32. The method of claim 27, wherein said single peak wavelength is in a range from 560 to 580 nm.

33. The method of claim 27, wherein said single peak wavelength is in a range from 580 to 600 nm.

34. The method of claim 27, wherein said single peak wavelength is in a range from 600 to 620 nm.

35. The method of claim 27, wherein said single peak wavelength is in a range from 620 to 640 nm.

36. The method of claim 27, wherein said single peak wavelength is in a range from 640 to 660 nm.
